(19) United States
(12) Patent Application Publication
(10) Pub. No.: US 2001/0053848 A1
(43) Pub. Date: Dec. 20, 2001

(54) Hetero-oligomeric G protein-coupled receptors as novel drug targets

(76) Inventors: Yogesh C. Patel, Westmount (CA); Magalie Rocheville, Montreal (CA)

Correspondence Address: John K. McDonald, Ph.D., KILPATRICK STOCKTON LLP, 2400 Monarch Tower, 3424 Peachtree Road, N.E., Atlanta, GA, 30326, US

(21) Appl. No.: 09/802,758

(22) Filed: Mar. 9, 2001

Related U.S. Application Data
(60) Provisional application No. 60/188,203, filed on Mar. 10, 2000.

(51) Int. Cl. C12P 21/02; C12N 5/06; C07H 21/04

(52) U.S. Cl. 536/23.5; 435/69.1; 435/325

(57) ABSTRACT

The present invention provides hetero-oligomers of G protein-coupled receptors (GPCRs) as novel drug targets, cells expressing endogenous hetero-oligomers of GPCRs, and cells transfected with GPCRs that express GPCRs capable of forming hetero-oligomers of GPCRs. These cells are useful for evaluating the activity of known and putative receptor ligands including but not limited to drugs, hormones, neurotransmitters, cytokines and related agonists and antagonists.

### PRIOR RELATED APPLICATIONS

This application claims priority to U.S. provisional patent application serial No. 60/188,203 filed on Mar. 10, 2000.

### FIELD OF THE INVENTION

The invention relates to hetero-oligomeric G protein-coupled receptors (GPCRs) as novel drug targets, cells expressing endogenous hetero-oligomers of GPCRs, and cells transfected with GPCRs that express GPCRs capable of forming hetero-oligomers of GPCRs. These cells are useful for evaluating the activity of receptor ligands including but not limited to drugs, hormones, neurotransmitters, chemokines and related agonists and antagonists.

### BACKGROUND OF THE INVENTION

Diverse mediators comprising hormones, neurotransmitters, catecholamines, chemokines, eicosanoids, olfactory and photo-sensory stimuli exert their effects by activating G protein-coupled receptors (GPCRs). GPCRs, also known as seven-transmembrane helical receptors are a growing superfamily currently comprising over 500 glycoproteins in humans. These receptors transduce signals through the intracellular heterotrimeric GTP-binding proteins consisting of α, β, and γ subunits, in order to effect a variety of physiological responses in tissues and organisms.

A variety of ligands, such as neurotransmitters, peptides and proteins, bind to and activate GPCRs. Some ligands demonstrate anatomical and functional relationships. One non-limiting example is somatostatin (SST) and dopamine (DA). In the brain, the peptide SST is found in both interneurons as well as projection neurons in different regions, and is considered an important physiological regulator of motor, sensory, cognitive, limbic, and autonomic functions. The actions of SST are mediated by a family of GPCRs with five molecular subtypes, SSTR1 to SSTR5, that are widely distributed throughout the neuraxis, but with an especially rich concentration in the deeper cortical layers, the striatum, and most regions of the limbic system. Dopamine, like SST, acts through its own family of five GPCRs (D1R through D5R) which also display rich expression in the cerebral cortex, striatum, and limbic structures. Furthermore, the SSTR and DR families share about 30% sequence homology and appear to be structurally related.

Somatostatin (SST) and dopamine (DA), two major neurotransmitter systems in the brain and periphery, share a number of structural and functional characteristics. Several studies suggest that receptors for SST (SSTRs) and DA (DRs) are co localized in neuronal subgroups and that SST is involved in modulating DA-mediated control of motor activity in the striatum. There is considerable behavioral and clinical evidence for an interaction between the SST and DA systems (Havlick, E. K. et al. Pharmacol. Biochem. Behav. 4:455-459, 1976; Leblanc, R. et al. Neurology 38, 1887-1890, 1988). Intracerebroventricular (i.c.v.) injections of SST-14 in rats produce dose-dependent neurobehavioral changes progressing from hyperkinesia to catatonia (Havlick, E. K. et al. Pharmacol Biochem Behav. 4:455-459, 1976). In low doses, SST potentiates the behavioral hyperactivity of L-Dopa and produces excitation with stereotyped circular running behavior, whereas high doses produce muscle tremor and rigidity, catatonia, and barrel rotation. The clinically used SST analog octreotide, which interacts with three of the SSTR subtypes (SSTR2, SSTR3, SSTR5) produces a Parkinsonian syndrome when administered i.c.v. in monkeys, characterized by truncal ataxia and severe hypokinesia reversible by subcutaneous injections of the DA agonist apomorphine (Leblanc, R. et al. Neurology 38, 1887-1890, 1988). The dual excitatory and inhibitory effects at the D2R occur through differential activation of either post-synaptic receptors leading to increased movement, or of pre-synaptic receptors causing hypokinesia as a result of a reduction in DA release. Dopamine 2 receptor agonists increase the efficacy of SST for inhibiting growth hormone (GH) secretion in patients with GH-producing pituitary tumors. Central administration of DA activates both SST and SSTRs in the rat striatum (Izquierdo-Claros, R. M., Boyano-Adanez, M. C., Larsson, C., Gustavsson, L., Arilla, E. Brain Research Molecular Brain Research 47(1-2), 99-107 (1997).

Another non-limiting example of two transmitter systems that share anatomical and functional relationships are the somatostatin and opioid systems. Both systems use receptors that are GPCRs. Like somatostatin, the opioid system is widely expressed throughout the brain. Opioid receptors (ORs) are structurally related to SSTRs and also exist as membrane hetero-oligomers. SSTRs and ORs are colocalized in distinct neuronal subsets. Furthermore, somatostatin analogs display powerful analgesic properties through mechanisms that have never been understood. For instance, SST analogs relieve headaches within minutes in patients with acromegaly. The octapeptide analog octreotide relieves painful hypertrophic pulmonary osteoarthropy secondary to bronchial carcinoma. Intrathecal or epidural octreotide has powerful analgesic effects on post-operative and neoplastic pain. Octreotide has a morphine sparing effect. However, to date the molecular basis for the interaction between the SSTR and opioid receptor systems remains unknown.

Although different GPCRs may interact functionally through signaling at a post-receptor level, what is needed is new insight into direct protein-protein interaction between GPCRs on the plasma membrane. What is also needed is a convenient method and system for evaluating functional interaction between GPCRs on a cellular level. What is further needed is a convenient method and system for evaluating ligands suspected of possessing biological activity at a GPCR, in order to identify ligands that might be useful as drug candidates.

### SUMMARY OF THE INVENTION

The present invention provides hetero-oligomers of G protein-coupled receptors (GPCRs), wherein each hetero-oligomer comprises at least two different GPCRs. These hetero-oligomers provide the molecular basis for the interaction between GPCRs. The present invention provides a convenient method and system for evaluating functional interaction between GPCRs on a cellular level. The present invention also provides a convenient method and system for evaluating ligands suspected of possessing biological activity at a GPCR, in order to identify ligands that might be useful as drug candidates.

Each GPCR in the hetero-oligomer may belong to a different receptor family, or a different receptor subtype within the same receptor family. Each GPCR is associated with a different ligand, wherein at least two different receptor molecules form a hetero-oligomer of GPCRs. The present method provides a method for evaluating the putative activity of ligands, including but not limited to drugs, hormones, neurotransmitter, chemokines, and agonists and antagonists thereof, by exposing hetero-oligomers of naturally occurring or transfected GPCRs to the ligand being evaluated for suspected activity. Such ligand exposure may occur before, during or after exposure of the hetero-oligomer to another ligand which binds to one of the other GPCRs in the hetero-oligomer. The present invention also provides a method to determine the modulatory activity of known ligands for specific GPCRs within the hetero-oligomer by measuring the functional activity of one ligand before, during or after exposure of the hetero-oligomer to at least one other ligand.

This method may be employed using membranes of cells expressing hetero-oligomers of GPCRs. These cells may be transfected with GPCRs or may naturally express GPCRs. This method provides results concerning the ability of the ligand being evaluated to bind to a GPCR within the hetero-oligomer or to modulate the binding of another ligand with known affinity to one of the GPCRs in the hetero-oligomer.

The present invention provides transfected cells and transfected cell lines that express at least two GPCRs which form hetero-oligomers. These cells and cell lines are useful for evaluating putative ligands for activity on one or more than one GPCR in a hetero-oligomer. This method may also be employed using non-transfected cells expressing hetero-oligomers of GPCRs. These cells may be transfected with GPCRs or naturally express GPCRs. Intact cells are used to evaluate the functional activity of known or putative ligands by determining the ability of the ligand to affect various biological functions associated with GPCRs. Such biological functions include any biological function associated with GPCR activation, including but not limited to, adenylyl cyclase activity, conductance of voltage-gated calcium channels, activation of potassium channel currents, stimulation of mitogenesis, activation of MAP kinase pathways, receptor trafficking such as internalization and up-regulation, receptor desensitization, and induction of apoptosis. This method provides information concerning the ability of the putative ligand to affect biological functions associated with GPCRs and also to modulate the activity of another ligand with known ability to affect biological functions associated with GPCRs.

A ligand to at least one GPCR may bind to the hetero-oligomer for activating the function of the GPCR. This ligand is an agonist. A ligand to at least one receptor may bind to the hetero-oligomer for decreased functional activity of the GPCRs. This ligand is an antagonist. A combination of two different agonist ligands may bind to the hetero-oligomer to alter the functional activity of the new hetero-oligomer by enhancing activity. A combination of two antagonist ligands may bind to the hetero-oligomer to decrease functional activity of the new GPCR.

A first ligand to a first GPCR in the hetero-oligomer may modulate the activity of a second ligand to a second GPCR in the hetero-oligomer. Such activity may include binding and/or functional activity transduced through the GPCRs in the hetero-oligomer. The following are non-limiting examples of such modulation: a) if a first ligand to a first GPCR in the hetero-oligomer decreases the inhibitory functional activity of a second ligand to a second GPCR in the hetero-oligomer then the first ligand is an agonist to the hetero-oligomer; b) if a first ligand to a first GPCR in the hetero-oligomer decreases the stimulatory functional activity of a second ligand to a second GPCR in the hetero-oligomer then the first ligand is an antagonist to the hetero-oligomer; c) if a first ligand to a first GPCR in the hetero-oligomer increases the stimulatory functional activity of a second ligand to a second GPCR in the hetero-oligomer then the first ligand is an agonist to the hetero-oligomer; and d) if a first ligand to a first GPCR in the hetero-oligomer increases the inhibitory functional activity of a second ligand to a second GPCR in the hetero-oligomer then the first ligand is an antagonist to the hetero-oligomer. While not wanting to be bound by the following statement, it is believed that such inhibitory functional activity by an antagonist may be achieved in at least two ways, either by preventing the formation of oligomers or by promoting the formation of inactive dimers. Accordingly, the present invention provides for all combinations of ligands to the individual GPCRs within the hetero-oligomer. Such ligands may be agonists, antagonists, any modified agonist, any modified antagonist, or any combination thereof. The combinations of such agonists and antagonists will have different net effects on the functional activity of the hetero-oligomer as transduced through its constituent GPCRs.

Any combination of GPCRs or GPCR subtypes may form a hetero-oligomer of GPCRs in cells naturally expressing GPCRs or in cells transfected so as to produce GPCRs. All such hetero-oligomers of GPCRs are considered within the scope of the present invention. All such cells transfected so as to produce GPCRs or GPCR subtypes that form hetero-oligomers are considered within the scope of the present invention. The use of cells naturally expressing GPCRs and cells transfected so as to produce GPCRs to evaluate the activity of putative and known ligands of GPCRs is considered within the scope of the present invention.

All GPCRs associated as hetero-oligomers are considered within the scope of the present invention. For example, a first GPCR may be a member of the dopamine receptor family (DR1, DR2, DR3, DR4, or DR5) and a second GPCR may be a member of the somatostatin receptor family (SSTR1, SSTR2, SSTR3, SSTR4, or SSTR5). In another example, a first GPCR may be a member of opioid receptor family (δ, κ, μ) and a second GPCR may be a member of somatostatin receptor family (SSTR1-SSTR5). In yet another example, a first GPCR may be a member of dopamine receptor family (DR1-DR5) and a second GPCR may be a member of opioid receptor family (δ, κ, μ). In yet another example, a first GPCR may be a member of somatostatin receptor family (SSTR1-SSTR5) and a second GPCR may be a member of melanin-concentrating hormone receptor family (MCH).

The hetero-oligomer of GPCRs may be a dimer or a higher order oligomer. The combination of ligands may interact with a dimer or higher order oligomer. Furthermore, a single ligand is sufficient to trigger the formation of a hetero-oligomer. It is believed that a second ligand molecule interacts with the hetero-oligomer to stabilize the hetero-oligomer and to alter its functional activity.

The present invention provides transfected cells and transfected cell lines that express at least two GPCRs which form hetero-oligomers. The present invention provides cells expressing a hetero-oligomer of GPCRs. The present invention also provides a biophysical method, fluorescence resonance energy transfer (FRET) to determine if cells express hetero-oligomers of GPCRs.

The present invention provides a method to evaluate putative ligands for their ability to promote or inhibit the formation of active or inactive hetero-oligomers, the method comprising exposing the hetero-oligomer to a putative agonist or antagonist ligand and measuring effective FRET efficiency as an index of hetero-oligomer formation.

The present invention provides a method to evaluate putative ligands for agonistic or antagonistic functional activity at a hetero-oligomer of GPCRs, which comprises exposing the hetero-oligomer to the putative ligand and measuring the functional activity of the GPCRs, wherein an increased activity is indicative of an agonist and a decreased activity is indicative of an antagonist.

In accordance with the present invention there is provided a method of screening putative ligands for agonistic or antagonistic activity to a hetero-oligomer of GPCRs, which comprises the steps of: subjecting the hetero-oligomer to at least one putative agonistic or antagonistic ligand; and evaluating the effect of the putative agonistic or antagonistic ligand on functional activity of the hetero-oligomeric GPCRs, wherein an increased activity is indicative of an agonistic ligand or a decreased activity is indicative of an antagonistic ligand. The at least one ligand may be a combination of at least two agonists or mimetic molecules thereof. The at least one compound may be a combination of at least two antagonists or mimetic molecules thereof.

Information derived from study of the hetero-oligomers of GPCRs of the present invention is useful in designing new strategies for therapeutic intervention involving combinations of ligands such as drugs. The present invention demonstrates that one ligand can modify the efficacy of a second ligand through the hetero-oligomer of GPCRs. Enhancement of dopaminergic efficacy by somatostatin, for example by using SST agonists in combination with dopamine agonists to target a SSTR5/DR2 hetero-oligomer could produce enhanced dopaminergic neurotransmission for treatment of Parkinson's Disease. SST agonists could be combined with opioid compounds for treatment of pain disorders since somatostatin synergizes with the delta opioid receptor and mu opioid receptor. Such synergism by two agonists in combination will improve the efficacy of drug treatment with low individual doses of the agonists, thereby minimizing unwanted side-effects. Finally, one could target a somatostatin hetero-oligomer in cancer cells to induce apoptosis or a SSTR5/D2R hetero-oligomer e.g. pituitary tumors such as acromegaly and prolactinomas for more potent suppression of hormone hypersecretion as well as more potent tumor inhibitory effects.

Accordingly it is an object of the present invention to provide novel hetero-oligomers containing at least two GPCRs.

It is another object of the present invention to provide novel hetero-oligomers containing at least two GPCRs, wherein the GPCRs are SSTR and OR.

Yet another object of the present invention is to provide novel hetero-oligomers containing at least two GPCRs, wherein the GPCRs are SSTR and DR.

It is an object of the present invention to provide novel hetero-oligomers containing at least two GPCRs, wherein the GPCRs within the hetero-oligomer possess enhanced biological activity when compared to GPCRs not associated in a hetero-oligomer.

It is another object of the present invention to provide transfected cells expressing at least two different GPCRs.

It is another object of the present invention to provide transfected cells expressing at least two different GPCRs, wherein the GPCRs are SSTR and DR.

Yet another object of the present invention is to provide transfected cells expressing at least two different GPCRs, wherein the GPCRs are SSTR and OR.

Another object of the present invention to provide a transfected cell line expressing at least two different GPCRs.

Another object of the present invention is to provide transfected cells expressing at least two different GPCRs, wherein expression levels of each GPCR are within a physiological range.

Still another object of the present invention is to provide a method to determine if cells express hetero-oligomers of GPCRs.

Another object of the present invention is to provide a method to determine if cells express hetero-oligomers of GPCRs, wherein the method is fluorescence resonance energy transfer (FRET).

Yet another object of the present invention is to provide a system and method for evaluating putative ligands for GPCRs.

Another object of the present invention is to provide a system and method for evaluating putative ligands for their ability to bind to GPCRs in hetero-oligomers.

Still another object of the present invention is to provide a system and method for evaluating putative ligands for functional activity transduced through GPCRs in hetero-oligomers.

Another object of the present invention is to provide a system and method for evaluating putative ligands for their modulatory effects on the binding of other ligands to GPCRs in hetero-oligomers.

Still another object of the present invention is to provide a system and method for evaluating putative ligands for their modulatory effects on the functional activity of other ligands transduced through GPCRs in hetero-oligomers.

It is another object of the present invention is to provide a system and method for evaluating ligands as drug candidates.

One feature of the present invention is that the identification of hetero-oligomers of GPCRs, and the ligands that activate the hetero-oligomer comprising these GPCRs provides valuable information for combinations of ligands to be used in treating disease.

These and other objects, features and advantages of the present invention will become apparent after a review of the following detailed description of the disclosed embodiments.

### BRIEF DESCRIPTION OF THE DRAWINGS

FIG. 1 is a schematic representation of the functional hetero-oligomer formation of D2R and SSTR5 by complementation.

FIG. 2 is a schematic representation of the functional interaction of D2R with SSTR5.

FIG. 3 is a schematic representation of the effect of hetero-oligomer formation on agonist-induced receptor endocytosis.

FIG. 4A) illustrates the confocal fluorescence microscope images of CHO-K1 cells co expressing HA-SSTR5 and D2R; FIG. 4B) schematically represents a photobleaching experiment with CHO-K1 cells coexpressing HA-SSTR5 and D2R after treatment with SST-14 (10⁻⁶M).

FIG. 5 schematically represents the effective FRET-efficiency in the basal state as well as after treatment with SST-14 (10⁻⁶M), DA (10⁻⁴M), SST-14 (10⁻⁶M) and DA (10⁻⁴M), sulpiride (10⁻⁴M), eticlopride (10⁻⁴M) on plasma membrane of CHO-K1 cells co-expressing HA-SSTR5 and wild type (wt) D2R.

FIG. 6 schematically illustrates the ligand-dependent and dose-dependent hetero-oligomer formation of SSTR1 and SSTR5.

FIG. 7 schematically represents the functional interaction of a C-tail deletion mutant of hSSTR5 and delta opioid receptor (DOR) that form a hetero-oligomer.

FIG. 8 schematically represents the efficiency of G protein coupling in response to treatment with DOR agonists (DADLE, DPDPE), the DOR antagonist (Naltrindol), or the Mu opioid receptor agonist (DAMGO). G protein coupling was assessed by the ability of GTPγS to inhibit binding of the SSTR5 radioligand [¹²⁵I] LTT SST-28.

FIG. 9 schematically represents the response of different agonists and antagonists alone and in combination on adenylyl cyclase coupling by the DOR and SSTR5 hetero-oligomer assessed as the ability of the agonist or antagonist to inhibit forskolin-stimulated cAMP accumulation.

FIG. 10 schematically represents the effective FRET efficiency for the CHO-K1 cotransfectants in response to treatment with SST-14 (a SSTR5 agonist), DPDPE (a DOR agonist), or the two agonists in combination.

FIG. 11 schematically represents the FRET efficiencies for the endogenous hetero-oligomer studied in cultured primary rat cortical neurons. There was low basal FRET and a marked increase induced by SST-14 comparable to that induced by DADLE.

FIG. 12 schematically represents the FRET for endogenous hetero-oligomer studied in cultured primary rat striatal neurons. The neurons were treated with SST-14 and a marked FRET efficiency was obtained. There was low basal FRET and a marked increase induced by SST-14 comparable to that induced by DADLE.

FIG. 13 schematically demonstrate the effect of the SSTR subtype-selective agonists in inducing apoptosis in MCF-7 cells which endogenously express SSTR1, 2, 3 and 5.

### DETAILED DESCRIPTION OF THE INVENTION

Definitions

For the purpose of the present invention the following terms are defined below.

The term “hetero-oligomer” is intended to mean dimer, trimer, tetramer or higher order oligomer of GPCRs or GPCR subtypes.

The term “G protein-coupled receptor” (GPCR) is intended to mean any of the seven-transmembrane helical receptors of a superfamily of GPCRs. All GPCRs are considered within the scope of the present invention. The GPCRs transduce signals through the intracellular heterotrimeric GTP-binding proteins consisting of α, β, and γ subunits, in order to effect a variety of physiological responses in tissues and organisms. (Brann, M. R. (Ed.) Molecular Biology of G-Protein Coupled Receptors, Birkhauser, Boston, Mass. (1991))

The term “ligand” means any molecule that binds to a receptor. A ligand may be an agonist, an antagonist or exhibit both agonistic and antagonistic behavior with respect to a receptor. Ligands may be any endogenously occurring molecule or derivative thereof, or any synthetic molecule or derivative thereof that binds to a receptor. Ligands may include but are not limited to drugs, chemicals, hormones, neurotransmitters, cytokines, and agonists and antagonists thereof.

The term “functional activity” means any biological activity associated with a GPCR or with hetero-oligomers comprised of GPCRs.

G-protein Coupled Receptors and Hetero-oligomer Formation

All GPCRs are considered within the scope of the present invention as constituents of hetero-oligomers. It is believed that any combination of GPCRs could form hetero-oligomers. These GPCRs include but are not limited to the following, somatostatin, opioid, adrenergic, adenosine, cannabinoid, dopamine, endothelin, muscarinic, serotonin, chemokine, melanocortin, neuropeptide Y (NPY), GnRH, GHRH, GHRP, TSH, LH, FSH. Other GPCRs, and some of the ligands that affect them are described in Trends in Pharmacological Sciences: Ion Channel Nomenclature Supplement compiled by S. P. H. Alexander & J. A. Peters, 11^{th} edition, Current Trends, London, UK, 2000, and The RBI Handbook of Receptor Classification and Signal Transduction, K. J. Watling, J. W. Kebabian, J. L. Neumeyer, eds. Research Biochemicals International, Natick, Mass., 1995, which are hereby incorporated by reference in their entirety.

Some preferred pairs of receptors for hetero-oligomer formation include but are not limited to dopamine and somatostatin receptors, somatostatin and opioid receptors, opioid and dopamine receptors, dopamine and serotonin receptors, NPY and melanocortin receptors, GHRH and GHRP receptors, dopamine and LHRH receptors, NPY and adrenergic receptors, NPY and serotonin receptors, TSH and LH receptors, TSH and FSH receptors, LH and FSH receptors, and LHRH and adrenergic receptors. It is to be understood that other pairs of GPCRs may form hetero-oligomers, and these GPCRs and the ligands that may stimulate hetero-oligomer formation by these GPCRs may be found in the references by Watling et al., and Alexander & Peters cited in the preceding paragraph.

Hetero-oligomers of GPCRs may also form between receptor subtypes within and between different receptor families. Within a receptor family, dopamine receptor subtypes may form a hetero-oligomer. Other non-limiting examples of hetero-oligomers that may form between receptor subtypes within a receptor family include the somatostatin receptor family, the opioid receptor family (Delta, Mu, and kappa receptors), the serotonin receptor, the melanocortin receptor family, the chemokine receptor family, the NPY family, the GHRH receptor family, the GHRP receptor family, and the adrenergic receptor family. The present invention demonstrates that dopamine (DA) receptors (DR) and somatostatin (SST) receptors (SSTR) families interact physically through hetero-oligomer formation to create a novel receptor with enhanced functional activity. The data demonstrate that D2R and SSTR5 receptor subtypes interact physically through hetero-oligomer formation to create a novel receptor with enhanced functional activity. The present invention further demonstrates that opioid receptors (ORs) and SSTR families interact physically through hetero-oligomer formation to create a novel receptor with enhanced functional activity. These data provide the first evidence that receptors from different GPCR families interact through hetero-oligomer formation. Such direct intramembrane association defines a new level of molecular cross talk between related GPCR subfamilies.

Cells

A variety of cells may be transfected with GPCRs in order to produce expression of hetero-oligomers of GPCRs. It is to be understood that the present invention is not limited to any specific cell type. All cells that may be transfected to express GPCRs are considered to be within the scope of the present invention.

Other cells that endogenously express hetero-oligomers of GPCRs in the absence of transfection may also be used in the context of the present invention to evaluate the activity of a putative ligand such as a drug, transmitter, hormone, chemical or other compound. Many endocrine, neural, neuroendocrine, immune, stem, fetal and tumor cells express endogenous receptors. All of these cells are useful for evaluation of their endogenous receptors to determine if they form hetero-oligomers and display pharmacological properties different from any of the individual receptors. Non-limiting examples of cells that contain endogenous SSTRs useful for these kinds of studies are neonatal cells, pituitary cells, islet cells, neuronal cells, neuroendocrine cells, immune cells, endocrine cells, tumor cells, acinar cells and other cell lines. Specific cells include but are not limited to the following: AtT-20 mouse pituitary cells; GH₃ and GH₄C₁ rat pituitary cells (which also express dopamine receptors); PC12 rat pheochromocytoma cells; RinM5F islet insulinoma cells; neuro2A neuroblastoma cells; human MCF-7 breast cancer cells (which express four of the somatostatin receptors); AR42J rat pancreatic acinar cells (rich in SSTR2); and Jurkatt and other human leukemic and myeloma cell lines.

Furthermore, it is to be understood that cells that endogenously express one or more GPCRs may also be transfected with one or more GPCRs. Such cells are useful to study the interactions of expressed GPCRs and to test putative ligands for activity.

Preferred cells for transfections with GPCRs are CHO-K1 (Chinese hamster ovary) cells, HEK-293 (human embryonic kidney) cells, MCF-7 cells and COS-7 monkey kidney cells HEK-293 cells are beneficial since they are of human origin and human somatostatin receptor and human dopamine receptor genes are readily available. HEK-293 cells are widely used in many different laboratories, transfect well and yield relatively high levels of expression of receptors. We have determined that host untransfected HEK-293 cells express low levels of some of the SSTR subtypes endogenously.

CHO-K1 cells do not appear to express endogenous receptors. A preferred cell line for transfection and expression of hetero-oligomers of GPCRs is the CHO-K1 cell.

MCF-7 human breast cancer cells are another preferred cell for investigation of hetero-oligomers of SSTR and their subtypes, or hetero-oligomers of SSTR with other GPCR receptors. MCF-7 cells express SSTRs. The MCF-7 cell is particularly useful for studying the cytostatic and cytotoxic (apoptotic) effects of somatostatin. Somatostatin itself is a known anticancer agent and is used for treating pituitary tumors as well as neuroendocrine tumors of the pancreas and intestine, especially carcinoid, VIPoma, glucagonoma, and others. Somatostatin acts through at least four of the receptors, SSTR1, SSTR2, SSTR4, and SSTR5 to induce cell growth arrest (cytostasis) and uniquely acts on SSTR3 to induce cell killing via apoptosis. The somatostatin analogs octreotide, sandostatin LAR, and lanreotide are available clinically for these purposes. Other commercially available compounds may be used as selective peptide agonists as well as nonpeptide agonists which show selectivity for individual subtypes. These could be used for enhancing the antiproliferative effects of somatostatin. In the case of hetero-oligomers, we have observed that although SSTR3 is the only receptor that induces apoptosis when this receptor is expressed by itself, in those cells where SSTR3 is co expressed with other SSTRs, those other SSTRs can also be activated to induce apoptosis through the formation of hetero-oligomers with SSTR3. We have also demonstrated this result in MCF-7 human breast cancer cells which express multiple SSTR subtypes.

Ligands

Any ligand specific for a GPCR may be used to affect the GPCR, and may induce hetero-oligomer formation. Endogenous naturally occurring and synthetic ligands specific for different GPCRs are well known to one of ordinary skill in the pharmacological sciences. Such ligands are described in Trends in Pharmacological Sciences: Ion Channel Nomenclature Supplement compiled by S. P. H. Alexander & J. A. Peters, 11^{th} edition, Current Trends, London, UK, 2000, and The RBI Handbook of Receptor Classification and Signal Transduction, K. J. Watling, J. W. Kebabian, J. L. Neumeyer, eds. Research Biochemicals International, Natick, Mass., 1995.

The endogenous ligands for the SSTRs include SST-14, SST-28, cortistatin-14, and cortistatin-29. The endogenous ligand for the dopamine receptors is dopamine. The endogenous ligand for the serotonin family of receptors is serotonin. The endogenous ligands for the melanocortin receptors are ACTH and α-MSH. The endogenous ligands for the NPY family of receptors is NPY and peptide YY. The endogenous ligands for the glycoprotein hormone receptors are LH and HCG for the LH receptor, FSH for the FSH receptor, and TSH for the TSH receptor. The endogenous ligand for the GHRH receptor is GHRH. The endogenous ligand for the GHRP receptor is a peptide known as GHRELIN.

Methods of Transfecting

Standard techniques known to one of ordinary skill in the art are employed for performing transfections. Such methods include but are not limited to the following, lipofectamine, calcium phosphate precipitation, electroporation, gene guns, liposomes, viral vectors (SV 40, baculovirus, vaccinia virus, and retroviruses. Lipofectamine and calcium phosphate precipitation are the generally preferred methods for transfections of GPCRs into cells.

For example, in the case of mono or cotransfection of CHO-K1 cells, the cells are grown to approximately 60-80% confluency in plastic Petri dishes and transfected with approximately 1 to 15, more preferably 3 to 10 μg, most preferably 5 μg of plasmid DNμg of one or both genes encoding for GPCRs using the Lipofectamine Plus reagent (provided by Gibco BRL) according to the manufacturer's instructions. The cells are grown in the presence of antibiotic using recommended concentrations. Following transfection with nucleic acid sequences encoding for a GPCR, cells are plated into multiple wells each containing only a few cells. These cells are permitted to grow and are next evaluated for binding of at least one ligand specific for at least one of the GPCRs expressed in the transfected cell. Those cells possessing the desired level of binding, which is indicative of the desired level of expression, are propagated further. In this manner, cells with low, medium, or high levels of GPCR receptor expression may be harvested and propagated further. Low, medium and high levels of GPCR receptor expression are defined relative to levels of binding of ligands commonly employed by one of skill in the art to measure binding of ligands to specific GPCR receptors. Preferred levels of GPCR expression are within a physiological range. Desired ranges of receptor expression are 100-400 fmol/mg protein.

Methods of Screening Compounds for Binding Activity to the Hetero-oligomers of GPCRs

Cells expressing endogenous hetero-oligomers of GPCRs and also transfected cells expressing hetero-oligomers of GPCRs may be investigated for the amount of expressed GPCRs using techniques known to one of ordinary skill in the art of conducting binding assays. Furthermore, binding assays may be used to analyze the ability of a first ligand to modulate binding of a second ligand to the hetero-oligomer.

Typically, membranes containing GPCRs are isolated from cell homogenates and examined using known saturation and displacement methods. Membranes are placed in a suitable container, for example a test tube, at a desired total protein concentration, and then exposed to a labeled ligand specific for the receptor being investigated. In some cases increasing amounts of labeled ligand are added to different containers containing the membranes and the extent of binding of labeled ligand to the membranes is measured. By analyzing the amount of binding of ligand to the membranes, one of ordinary skill in the art may calculate the receptor density, usually expressed in terms of the amount of receptors per amount of membrane protein.

In other cases, a constant amount of labeled ligand is added to the different containers containing similar amounts of membranes. Next, increasing concentrations of a ligand which displaces the labeled ligand from the GPCR are added to the different containers. By analyzing the amount of labeled ligand remaining on the membranes as a function of dose of competing unlabeled ligand, one of ordinary skill in the art may calculate the Ki values and the IC₈₀, IC₅₀, IC₂₀ or any other amount of competing ligand that displaces various percentages of labeled ligand from the receptor. Both agonist ligands and antagonist ligands may be revealed using these membrane binding studies. Any label may be bound to a ligand, including but not limited to radiolabels, colorimetric labels, fluorescent labels and any other label known to one of ordinary skill in the art.

Using these methods, one may quantitatively assess the level of expression of one or more GPCRs in a cell. This assessment provides a means to evaluate the efficacy of transfections of a cell with one or more GPCRs. The present inventors have observed that levels of expression of GPCRs that are within a relatively physiological range are preferred for analysis of hetero-oligomers. One of ordinary skill in the art is familiar with a physiological range for each GPCR or GPCR subtype. For example, for the somatostatin receptor (SSTR), such a range is about 100-400 fmol/mg protein (Srikant and Patel, Proc. Natl. Acad. Sci. USA 78(6) 3930-3934 (1981); Rocheville, M. et al., J. Biol. Chem. 275:7862-7869 (1999).

The Ki and IC₈₀, IC₅₀, IC₂₀ and similar data concerning displacement or inhibitory efficacy of a competing ligand are employed in selecting dosages of such ligand for use in studying functional activity of hetero-oligomers of GPCRs. One may investigate the ability of a first ligand to modify the binding of a second ligand to a hetero-oligomer. Such studies may be performed by selecting an appropriate dosage or dose range of a first ligand and examining its ability to modify binding of a second ligand to the hetero-oligomer at Ki, IC₈₀, IC₅₀, IC₂₀ values for the second ligand. Alternatively, a fixed concentration of a first ligand may be used to examine its effect on enhancing or reducing the binding of a second ligand throughout a dose range of the second ligand. Such competition binding paradigms are known to one of ordinary skill in the art.

Ligands and labeled ligands useful in studying GPCRs are known to one of ordinary skill in the art and can be prepared as radio-iodinated ligands as described (Srikant and Patel, Proc. Natl. Acad. Sci. USA 78(6) 3930-3934 (1981); or obtained commercially from vendors such as SIGMA, Aldrich, Amersham, Calbiochem, Phoenix Pharmaceuticals, Molecular Probes and Pharmingen. Ligands and labeled ligands useful in studying GPCRs are described in Trends in Pharmacological Sciences: Ion Channel Nomenclature Supplement compiled by S. P. H. Alexander & J. A. Peters, 11^{th} edition, Current Trends, London, UK, 2000, and The RBI Handbook of Receptor Classification and Signal Transduction, K. J. Watling, J. W. Kebabian, J. L. Neumeyer, eds. Research Biochemicals International, Natick, Mass., 1995.

In one embodiment concerning SSTRs, binding studies are generally performed on cell membranes prepared from transfected cells by homogenization followed by centrifugation. Binding studies are then carried out for about 30 min at 37° C. with about 20-40 μg of membrane protein and [¹²⁵I]-LTT SST-28 (in the case of SSTRs) in about 50 mM HEPES KOH buffer pH 7.5 containing approximately 5 mM Mg²⁺, 0.02% BSA, 200 kallikrein inhibitor units/ml aprotinin, 0.02 μg/ml phenylmethylsulfonyl fluoride, and 0.02 μg/ml Bacitracin. Incubations are terminated by the addition of about 1 ml HEPES KOH containing 0.2% BSA, rapid centrifugation, and washing. Radioactivity associated with membrane pellets is counted in a gamma counter. Specific binding is defined as the difference between counts bound in the absence and presence of 100 nM SST. Saturation binding experiments are performed with membranes using increasing concentrations of radioligand (2-2000 picomoles (pm)) under equilibrium binding conditions. Competition analyses are carried out through incubation of membranes with radioligand (approximately 60 pm) and increasing concentrations of test substances. Binding data are analyzed with Prism 3.0 (GraphPad Software, San Diego, Calif.). This approach may be readily adapted by one of ordinary skill in the art to study other receptors or their subtypes.

Functional Assessment of Hetero-oligomers

Several different assays may be employed to study GPCRs and the various signal transduction mechanisms associated with GPCRs. GPCRs are known to affect adenylyl cyclase activity, to modulate the conductance of voltage-gated calcium channels, to modulate potassium channel currents, especially inward rectifying K⁺channels, to activate the MAP kinase pathway, to activate the phospholipase C (PLC)-IP₃ pathway, to activate phosphotyrosine phosphatase, to stimulate mitogenesis, to stimulate exocytosis, to stimulate cytostasis, to stimulate chemotaxis and to induce apoptosis. Techniques for performing these measurements are commonly known to one of ordinary skill in the art.

These assays may be employed to examine the functional activity of hetero-oligomers of GPCRs. The functional activity of hetero-oligomers may be assessed by examining the effects of a first ligand specific for a first GPCR in the hetero-oligomer on the efficacy of a second ligand specific for the second GPCR in the hetero-oligomer. First ligands may be an endogenous or naturally occurring ligand, for example a catecholamine such as norepinephrine or dopamine. First ligands may also be an agonist or antagonist, natural or synthetic. Such ligands are known to one of skill in the art of receptor pharmacology. Representative ligands are found in Trends in Pharmacological Sciences: Ion Channel Nomenclature Supplement compiled by S. P. H. Alexander & J. A. Peters, 11^{th} edition, Current Trends, London, UK, 2000, and The RBI Handbook of Receptor Classification and Signal Transduction, K. J. Watling, J. W. Kebabian, J. L. Neumeyer, eds. Research Biochemicals International, Natick, Mass., 1995.

The following examples are illustrative embodiments of the invention, not limiting the scope of the invention in any way. It will be appreciated that other embodiments and uses will be apparent to those skilled in the art and that the invention is not limited to these specific illustrative examples.

### EXAMPLE 1

Cotransfection of CHO-K1 Cells

In the case of mono or cotransfection of CHO-K1 cells, the cells were grown to about 70% confluency in 100 mm diameter plastic Petri dishes and transfected with approximately 5 μg of plasmid DNA of one or both genes encoding for GPCRs using the Lipofectamine Plus reagent (provided by Gibco BRL) according to the manufacturer's instructions. The cells were grown in the presence of the antibiotic G418 (700 μg/ml) and after transfections, replated cells into multiple wells each containing only a few cells. These cells were permitted to grow and were next evaluated for binding. The wells with the desired level of binding were selected for further propagation. In this way because cells will transfect at different levels or not at all, we can harvest cells with low, medium, or high levels of expression.

### EXAMPLE 2

Characterization of the Anatomical Distribution Between the Long Form of the Human (h) D2R and hSSTR5

hSSTR5 is a SSTR subtype previously reported to undergo ligand-dependent hetero-oligomer formation and to form hetero-oligomers with other SSTRs (hSSTR1) (Rocheville, M., et al., J. Biol. Chem. 275:7862-7869 (1999). Likewise, DRs have been shown to exist as oligomers on the plasma membrane (Izquierdo-Claros, R. M., et al., Brain Research. Molecular Brain Research 47(1-2), 99-107 (1997). Both receptors signal through inhibition of adenylyl cyclase via Gi proteins (Patel, Y. C. Frontiers in Neuroendocrinology 20:157-198 (1999); Missale, C., et al., Physiological Review 78 (1): 189-225 (1998). By immunocytochemistry, D2R and SSTR5 were colocalized in distinct neuronal subsets in striatum and cerebral cortex using peroxidase and fluorescence immunocytochemistry.

Adult male CD rats were anesthetized with ketamine, perfusion fixed and 40 μM coronal sections of brain processed for double-label immunocytochemistry. Antipeptide antibodies against the amino terminal segment of hSSTR5 or residues 231-244, the third extracellular loop of hD2R, were produced in rabbits, validated by Western blots and used as primary antibodies. Sections were incubated with SSTR5 antibody (1:500) overnight followed by incubation with FITC conjugated anti-rabbit IgG (1:100). The same sections were then exposed to D2R antibody (1:300) overnight and receptor was visualized with Cy3 conjugated anti-rabbit IgG (1:300). Since both primary antibodies were anti-rabbit, the specificity of the immunofluorescent co localization was validated by exposure of the SSTR5 labeled brain sections with the SSTR5 peptide immunogen for 3-4 h at room temperature prior to incubation with D2R antibody. Co-expression of SSTR5 and D2R was also demonstrated independently in serial sections developed for SSTR5 and D2R localization by peroxidase immunocytochemistry. Many neurons expressed D2R in serial sections of rat striatum. A subset of these neurons, with the morphological characteristics of medium aspiny neurons, showed co-expression of SSTR5. Co-localization of SSTR5 and D2R was confirmed by indirect immunofluorescence. Analysis of confocal images of striatal and cortical regions showed neurons double-labeled for D2R and SSTR5 by immunofluorescence. D2R was localized by Cys imaged in red fluorescence. SSTR5 positive neurons were localized by FITC imaged in green and identified in the same section. Co-expression of D2R with SSTR5 were observed by the yellow orange color in the merged images.

D2R was expressed in many neurons whereas SSTR5 immunoreactivity is restricted to a selective neuronal subpopulation (data not shown). Most of these neurons displayed morphological characteristics of medium aspiny neurons and show strong co localization with D2R in adjacent sections as well as in overlapped confocal images. Similar co localization of SSTR5 and D2R was observed in cerebral cortex. By double label confocal fluorescence microscopy, SSTR5 immunoreactivity was expressed in pyramidal neurons mainly in the deeper cortical layers whereas D2R occurred in both pyramidal and nonpyramidal neurons in all cortical layers. SSTR5 displayed strong co-expression with D2R in cortical pyramidal neurons.

### EXAMPLE 3

Hetero-oligomer Formation by D2R and SSTR5 Receptor Subtypes in a Heterologous System: Effect of Stably Cotransfecting D2R and delta318-SSTR5 Mutants on Ligand-induced Adenylyl Cyclase Coupling.

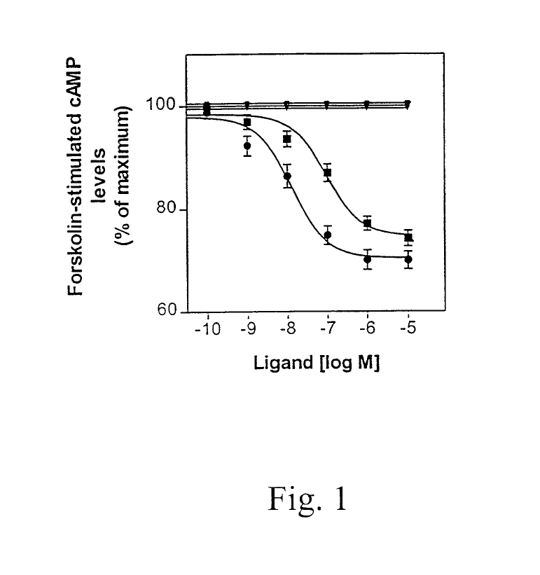

Having found endogenous co-expression of D2R and SSTR5, we investigated hetero-oligomer formation by the two subtypes in an heterologous system by functional rescue of a partially active C-tail deletion mutant of human SSTR5 (delta318 SSTR5) previously characterized (Hukovic, N. et al., J. Biol. Chem. 273 (33): 21416-21422 (1998). This mutant displays complete loss of adenylyl cyclase coupling whilst retaining full agonist binding potency. We investigated whether loss of adenylyl cyclase coupling could be rescued by co-transfection with D2R whereby the mutant receptor would associate with and signal through the C-tail of D2R. Delta318 SSTR5 was stably expressed in CHO-K1 cells either alone or with D2R. As reported previously the delta318 SSTR5 mutant showed complete loss of the ability to inhibit forskolin-stimulated cAMP levels upon SST treatment (FIG. 1).

Receptor coupling to adenylyl cyclase was tested by incubating cells for 30 min with 1 μM forskolin with or without SST (10⁻¹⁰-10⁻⁶ M) at 37° C. as previously described (Hukovic, N., et al., J. Biol. Chem. 273 (33): 21416-21422 (1998). Delta318-hSSTR5 monotransfectants treated with SST-14 (inverted solid triangles) were compared with cotransfectants treated with SST-14 (solid squares) or DA (solid circles). SST-14 produced dose-dependent inhibition of forskolin-stimulated cAMP which was completely abolished by 100 ng/ml pertussis toxin pretreatment for 2 h (inverted solid triangles) or by addition of the DA antagonist sulpiride (10⁻⁴ M) (inverted solid triangles). Data shown are mean+SEM of 3 experiments. When co-expressed with D2R, however, SST induced dose-dependent inhibition of cAMP levels by the cotransfectants to a maximum of 26±1.5% at 10⁻⁶ M which was completely abolished by pertussis toxin treatment. Such functional complementation suggests that the delta318 SSTR5 and D2R receptors associate as hetero-oligomers to constitute a functional Gi protein linked effector complex. Treatment of the cotransfectants with DA produced 30±1.7% reduction in cAMP presumably through a direct effect of the agonist on the wild type D2R. Addition of sulpiride (10⁻⁴ M) a DA receptor antagonist, however, completely abolished the ability of SST to inhibit forskolin-stimulated cAMP by delta318 SSTR5-D2R putative hetero-oligomers (FIG. 1). Such differential ability of agonist- or antagonist-bound D2R to form hetero-oligomers with delta318 SSTR5 could be explained by different conformational states of the agonist- or antagonist-occupied receptor.

### EXAMPLE 4

Interaction Between SSTR5 and D2R on Agonist Binding Affinity, G Protein and Adenylyl Cyclase Coupling using Transfected Wild Type Receptors

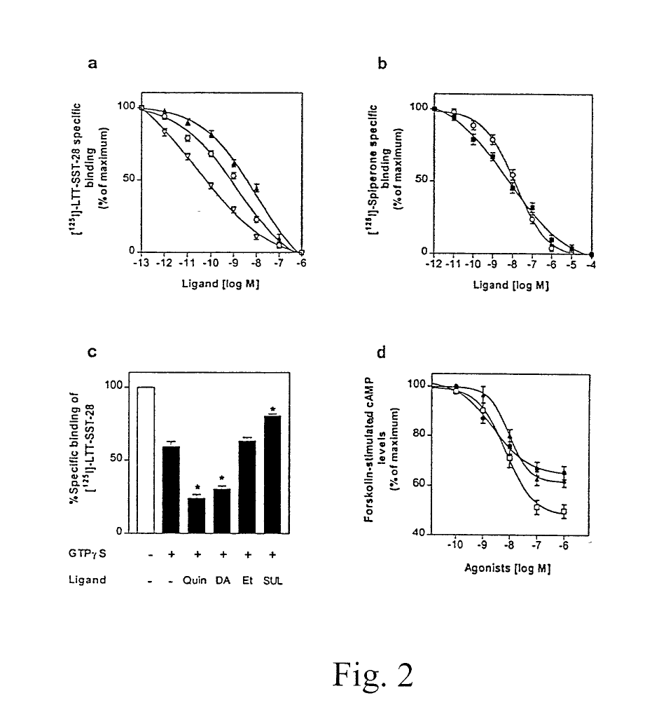

Human SSTR5 tagged at the amino terminus with a nonapeptide of the hemagglutinin protein (HA-SSTR5) and D2R were stably cotransfected in CHO-K1 cells to achieve levels of expression (Bmax 163±22 fmol/mg protein for HA-SSTR5, 107±29 fmol/mg protein for D2R) comparable to the density of endogenous receptor expression (Patel, Y. C. Frontiers in Neuroendocrinology 20:157-198 (1999); Missale, C., et al., Physiological Review 78 (1): 189-225 (1998)). FIG. 2a. Displacement analysis using the SSTR-specific [¹²⁵I]-LTT-SST-28 radioligand. Membranes co-expressing HA-SSTR5 and D2R were treated with increasing amounts of SST-14 alone (open circles), SST-14 and quinpirole (10⁻⁴ M) (open triangles), or SST-14 and sulpiride (10⁻⁴ M) (solid triangles). D2R ligand concentrations were selected to reach saturation binding, as well as maximal signaling (quinpirole) or inhibition of signaling (sulpiride). FIG. 2b. Displacement analysis using the D2R-specific [¹²⁵I]-Spiperone radioligand. Membranes co-expressing HA-SSTR5 and D2R were treated with increasing amount of sulpiride alone (open circles), or sulpiride and SST-14 (10⁻⁶ M) (closed squares). The SSTR agonist concentration was selected to ensure maximal saturation binding and receptor signalling. FIG. 2c. G-protein coupling of the expressed receptors was assessed by investigating the effect on [¹²⁵I]-LTT-SST-28 binding of incubating membranes from cotransfectants with GTPγS (10⁻⁴M) for 30 min at 37° C. Maximum specific binding obtained in the absence of GTPγS (clear bar) was about 15%. SST-14 (10⁻⁶ M) was used to define non-specific binding. GTPγS treatment significantly reduced specific binding of [¹²⁵I]-LTT-SST-28 to HA-SSTR5, reflecting G-protein coupling. This response was enhanced by addition of quinpirole (10⁻⁴ M) and DA (10⁻⁴ M), but was not affected by eticlopride (10⁻⁴ M), but was reduced by sulpiride (10⁻⁴ M). Ligand concentrations used were chosen to reach saturation binding. FIG. 2d. Receptor coupling to adenylyl cyclase (Rocheville, M., et al., J. Biol. Chem. 275:7862-7869 (1999) was assessed as inhibition of forskolin stimulated cAMP following treatment of the co-transfectants with SST-14 (open circles), quinpirole (closed triangles), or SST-14 and quinpirole (open squares). Mean+SEM of 3 independent experiments. *, p<0.01 compared with control GTPγS-treated (Dunnett's post hoc one-way analysis of variance).

Using [¹²⁵I] LTT SST-28 as the SSTR radioligand, competition analysis showed a 30 fold increase in the binding affinity of SST-14 upon addition of the D2R agonist quinpirole (10⁻⁴ M) (from Ki 1.53±0.2 nM to Ki 0.05±0.01 nM) (FIG. 2a). In contrast, addition of the D2R antagonist sulpiride caused a 5-fold reduction in the inhibitory potency of SST-14 for binding to the putative HA-SSTR5/D2R dimers (from Ki 1.53±0.2 nM to Ki 7.46±1.2 nM) (FIG. 2a). Since neither the DA agonist nor antagonist is capable of binding to HA-SSTR5 directly, these results further suggest that the binding affinity of HA-SSTR5 for SST-14 is modulated by different conformational states of the agonist- or antagonist-occupied D2R through HA-SSTR5/D2R hetero-oligomers. Converse findings were also obtained in the case of the effect of SST on the binding affinity of the D2R (FIG. 2b). Displacement analysis of [¹²⁵I] spiperone binding by sulpiride showed a modest 2 fold increase in the inhibitory potency of sulpiride for the D2R in the presence of SST especially at lower concentrations (from Ki 17.2±2.6 nM to Ki 8.2±1.4 nM). This suggests a synergistic role of SST on D2R affinity perhaps at low but not high concentrations due to a more suitable conformation for [¹²⁵I] spiperone binding by HA-SSTR5/D2R hetero-oligomers.

G protein coupling of HA-SSTR5 and D2R was assessed by monitoring the effect of GTPγS treatment on membrane [¹²⁵I] LTT SST-28 binding (FIG. 2c). GTPγS treatment of the cotransfectants led to a 41±3% decrease in specific radioligand binding. Both DA and the DA agonist quinpirole increased inhibition by GTPγS. The DA antagonist displayed no effect on SST-14 induced G protein coupling of HA-SSTR5 whereas sulpiride significantly reduced inhibition by GTPγS. Treatment of the cotransfectants with SST-14 or quinpirole induced maximum 36±3% and 39±3% inhibition of forskolin stimulated cAMP respectively (FIG. 2d). Simultaneously application of both agonists potentiated the cAMP inhibitory response to a maximum of 52±4%. Sulpiride reduced SST-14 induced cAMP inhibitory response (maximum inhibition 25±2%) and acted as a partial antagonists of SST-14 signaling.

### EXAMPLE 5

The Effect of Co-expressing D2R with SSTR5 on the Process of Receptor Internalization

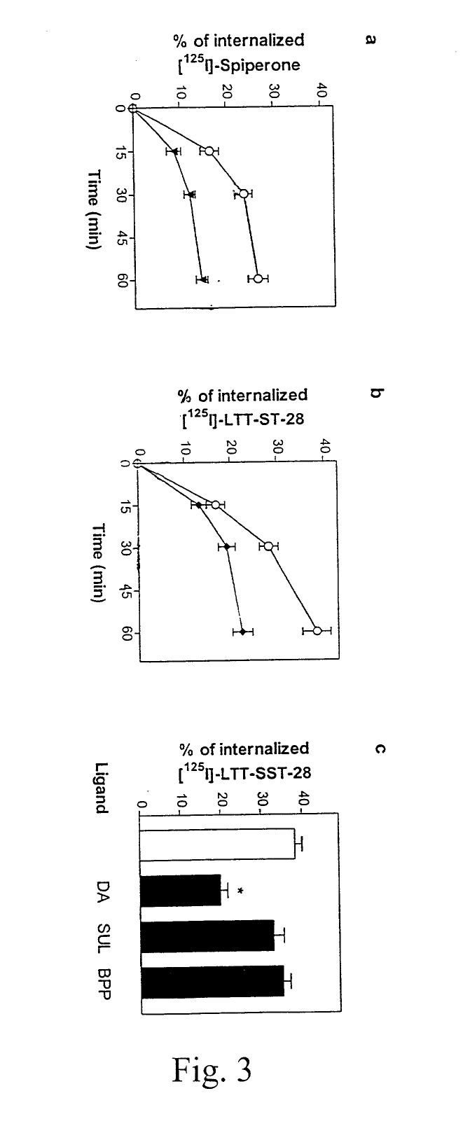

Internalization experiments were carried out by incubating cells overnight at 4° C. with radioligand with or without cold ligand as previously described (Hukovic, N., et al., J. Biol. Chem. 273 (33): 21416-21422 (1998). FIG. 3a. Percent internalization of [¹²⁵I]-Spiperone by HA-SSTR5/D2R cotransfectants in the presence (solid inverted triangles) or absence (open circles) of SST-14 (10⁻⁶ M). Sulpiride (10⁻⁴ M) was used to define non-specific binding. FIG. 3b. Percent internalization of [¹²⁵I]-LTT-SST-28 by HA-SSTR5/D2R cotransfectants in the presence (closed circles) or absence (open circles) of quinpirole (10⁻⁴ M). SST-14 (10⁻⁶ M) was used to define non-specific binding. FIG. 3c. Maximum internalization of [¹²⁵I]-LTT-SST-28 HA-SSTR5/D2R cotransfectants after incubation at 37° C. for 60 minutes. Treatment with DA (10⁻⁴ M) significantly reduced internalization, whereas sulpiride (10⁻⁴ M), like the control peptide BPP (10⁻⁶ M), was without effect. SST-14 (10⁻⁶ M) was used to define non-specific binding. All of the ligand concentrations used were chosen to reach saturation binding. Results are mean±SEM of at least 3 experiments. *, p<0.01 compared with control non-treated (Dunnett's post hoc one-way analysis of variance).

Stable CHO-K1 cells co-expressing HA-SSTR5 and D2R were incubated at 37° C. for different times with [¹²⁵I] spiperone with or without 0.1 mM sulpiride. There was time-dependent internalization of radioligand with a maximum of 27±3% at 60 min (FIG. 3a). Treatment with SST-14 inhibited radioligand internalization to a maximum of only 14±2% at 60 min suggesting that the association of SSTR5 with D2R, while promoting ligand binding affinity and G protein coupling, impairs internalization of the hetero-oligomeric receptor complex (FIG. 3a). The SSTR radioligand [¹²⁵I] LTT SST-28 was also internalized in the cotransfected CHO-K1 cells in a time-dependent manner to a maximum of 38±3% at 60 min (FIG. 3b). Treatment with the D2R-specific agonist quinpirole attenuated [¹²⁵I] LTT SST-28 internalization to a maximum of 23±2.2% at 60 min (FIG. 3b). Similar 20±3.4% maximum internalization was observed with DA treatment (FIG. 3c). In contrast, the DA antagonist sulpiride or an unrelated peptide bovine pancreatic polypeptide (BPP) was without effect on internalization (maximum internalization of 33±4.4% and 36±2.2% respectively) (FIG. 3c).

### EXAMPLE 6

Direct Evidence for Association of SSTR5 and D2R in Intact Cells 7

In order to obtain direct evidence for association of SSTR5 and D2R in intact cells, we investigated receptor hetero-oligomer formation by photobleaching fluorescence resonance energy transfer (pbFRET) microscopy using known techniques (Rocheville, M. et al., J. Biol. Chem. 275:7862-7869 (1999).

Generally, FRET efficiencies are determined indirectly by measuring changes in the quantum yield of any competitive donor deactivation process upon introduction of an acceptor molecule. Donor photobleaching represents such a competitive process which is exploited in pbFRET-microscopy. The effective FRET efficiency E is calculated from the photobleaching time constants of the donor obtained in the absence (τ_{D-A}) and presence (τ_{D+A}) of acceptor according to:

$$E = 1 - \frac{{τ}_{D - A}}{{τ}_{D + A}} .$$

CHO-K1 cells, stably cotransfected with HA-hSSTR5 and D2R, were grown on glass coverslips for 24 hours, treated with various agonists/antagonists for 30 minutes at 37° C., washed and fixed in 1% paraformaldehyde for 30 minutes on ice. After incubation with 10% normal goat serum in phosphate buffered saline for 1 hour, HA-SSTR5 and D2R were specifically labeled with FITC (donor) and rhodamine (acceptor) using rabbit polyclonal antibodies (described in Rocheville et al., Science, pp. 154-157, Apr. 7, 2000), and mouse monoclonal anti-HA antibodies as primary antibodies followed by reaction with FITC or rhodamine conjugated secondary antibody. Coverslips were mounted on microscope slides and used for pbFRET-microscopy the day after.

pbFRET experiments were performed on a Leica DMLB fluorescence microscope equipped with epi-illumination. An OSRAM HBO 100 W Hg lamp was used as excitation light source and the objective was a Leica pl-fluotar 40× oil immersion lens. In order to separate FITC excitation from emission as well as to optimize FITC excitation while simultaneously blocking rhodamine excitation, the following filters were used: Leitz BP 450-490 (excitation), RKP510 (dichroic mirror), BP 515-535 (emission). Digital images (8-bit) were generated with an Electrim-1000U CCD camera with a spatial resolution of 1134×486 pixels of size 7.8×13.6 μM. For each cell, a sequence of 20 images was acquired during donor-photobleaching. One image was collected every 4 s with exposure time of 1 s. Exposure as well as time delays were software controlled.

Procedures for image analysis were written in-house using IGOR Pro 3.13 (Wavemetrics, Oregon). Images were corrected for dark current, background and flatness. For analysis of the photobleaching decay, only the high-intensity plasma membrane region was considered, low intensity background and intracellular regions were masked.

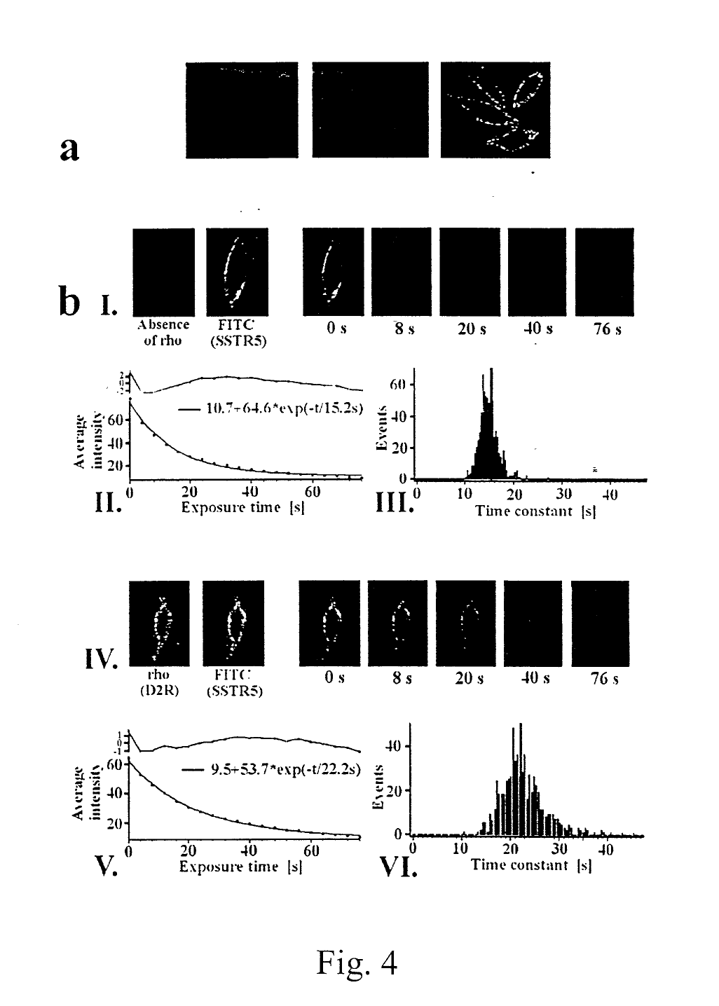

HA-hSSTR5 and D2R were specifically labeled with FITC (donor) and rhodamine (acceptor), respectively using mouse monoclonal antibody against HA and rabbit polyclonal anti-D2R antibody, followed by reaction with FITC conjugated goat anti-mouse and rhodamine conjugated goat anti-rabbit secondary antibody. Both reactions resulted in specific plasma membrane staining (FIG. 4A). FIG. 4A Left panel: HA-SSTR5 (visualized with mouse anti-HA mAb and FITC-conjugated goat anti-mouse IgG). FIG. 4A Middle panel: D2R (rabbit polyclonal antibody and rhodamine-conjugated goat anti-rabbit IgG). FIG. 4A Right panel: Colocalization of HA-SSTR5 and D2R (yellow). Magnification: ×630.

The decrease in donor fluorescence intensity due to photobleaching during prolonged exposure to excitation light was monitored in the absence (FIG. 4B, I-III) or presence (FIG. 4B, IV-VI) of acceptor, i.e. in the potential presence of an additional donor deactivation process, FRET. Cells were reacted with primary antibodies and either with FITC-conjugated goat-anti-mouse IgG (donor in the absence of acceptor) (I-III), or with both FITC-conjugated goat-anti-mouse and rhodamine-conjugated goat-anti-rabbit IgG (donor in the presence of acceptor) (IV-VI). FIG. 4B I. Photobleaching of donor in the absence of acceptor (only selection of images shown). For analysis of the photobleaching decay, only the high-intensity membrane region was considered, low intensity background and intracellular regions were masked (black). Leftmost: Unmasked images showing absence of rhodamine and initial donor fluorescence. FIG. 4B II. The average fluorescence intensity of each image was plotted versus the time that the cell had been exposed to excitation light. The monoexponential fit (red) as well as the residue (green) demonstrate the good approximation of the photobleaching decay by a single exponential. FIG. 4B III. The decrease of fluorescence intensity was analysed for each pixel of the unmasked region and fitted to a single-exponential decay. The resulting time constants were plotted in the histogram shown. The average time constant of 15.8 s (black bar) was taken as τ_{D-A}. FIG. 4B IV. Photobleaching of donor in the presence of acceptor. Leftmost: Unmasked images showing initial rhodamine and FITC-fluorescence. FIG. 4B, IV-VI. The presence of acceptor led to larger donor photobleaching time constants, with an average, τ_{D+A}, of 22.2 s, reflecting FRET between FITC and rhodamine.

The photobleaching decay was analyzed for the plasma membrane regions, both on a pixel-by-pixel basis (FIG. 4, III & VI) as well as averaged over each image (FIG. 4, II & V). Any slow-down of the photobleaching process (as described by an increase in the photobleaching time constant) upon addition of rhodamine labeled antibody suggests that a large proportion of rhodamine molecules are in close proximity to fluorescein to act as acceptors for energy transfer. Given that the two fluorophores are coupled to different receptor molecules, such slow-down indicates physical association of the receptors.

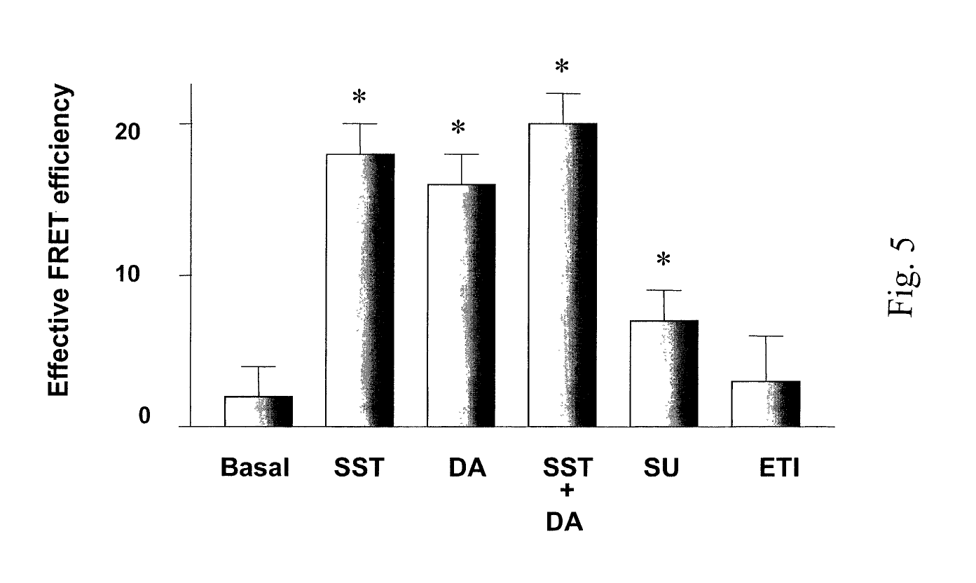

In the basal state, we found a low effective FRET efficiency of 2±2% reflecting an insignificant amount of preformed hetero-oligomers (FIG. 5 and Table 1).

TABLE 1
Measurement of effective FRET efficiency under various
treatment conditions

| Treatment | n | τ_{avg}[S] | σₙ₋₁[S] | E[%] |
|---|---|---|---|---|
| None | 48 | D + A: 18.3 ± 0.3 | 2.0 | 2 ± 2 |
| (basal state) | 45 | D − A: 17.9 ± 0.3 | 2.2 | |
| SST-14 | 45 | D + A: 21.8 ± 0.4 | 2.7 | 18 ± 2 |
| (10⁻⁴M) | 45 | D − A: 17.9 ± 0.3 | 2.2 | |
| DA | 50 | D + A: 21.3 ± 0.3 | 2.4 | 16 ± 2 |
| (10⁻⁴M) | 45 | D − A: 17.9 ± 0.3 | 2.2 | |
| SST-14 (1 μM) + | 36 | D + A: 22.4 ± 0.4 | 2.2 | 20 ± 2 |
| DA(10⁻⁴M) | 45 | D − A: 17.9 ± 0.3 | 2.2 | |
| Sulpiride | 58 | D + A: 19.2 ± 0.3 | 2.4 | 7 ± 2 |
| (10⁻⁴M) | 45 | D − A: 17.9 ± 0.3 | 2.2 | |
| Eticlopride | 48 | D + A: 19.4 ± 0.4 | 2.4 | 3 ± 3 |
| (10⁻⁴M) | 42 | D − A: 18.9 ± 0.3*⁾ | 2.2 | |

n: number of cells analyzed (with an average number of approx. 1500 pixels per cell); τ_{avg}: mean of n photobleaching time constants (each being the pixel-based average of a cell membrane), ± SEM; D − A, D + A: donor in absence and presence of acceptor; σₙ₋₁: standard deviation of τ_{avg}; E: average effective FRET-efficiency.
*⁾Absolute photobleaching time constants are not comparable between different date sets as they were measures on different days and were therefore affected by the decreased in excitation intensity of the UV lamp.

Treatment with either agonist SST-14 (10⁻⁶ M) or DA (10⁻⁴ M), resulted in a strong increase in FRET efficiency to 18±2% and 16±2% respectively, suggesting that formation of hetero-oligomers of SSTR5 and D2R is induced by either agonist (FIG. 5). Simultaneous treatment with both agonists (SST-14 10⁻⁶ M and dopamine 10⁻⁴ M) resulted in a similar FRET efficiency of 20±2%, indicating that a receptor-ligand stoichiometry of both 2:1 and 2:2 is possible. In contrast, treatment with the D2R-specific antagonists sulpiride (10⁻⁴ M) and eticlopride (10⁻⁴ M) led to a significantly lower FRET efficiency of 7±2% and 3±3% respectively. Ligand concentrations were selected to reach saturation binding, as well as maximum signalling (agonists) or maximum inhibition of signalling (antagonists). The number of cells analysed for each condition was approximately 45.

Using concurrent pharmacological and physical (FRET) methods of analysis, these results demonstrated that D2R and SSTR5 associate as functional hetero-oligomers on the plasma membrane and provide the first evidence that receptors from different GPCR families can interact through oligomer formation for greater functional diversity. The D2R/SSTR5 hetero-oligomer is pharmacologically distinct from its constituent GPCRs (D2R and SSTR5), being characterized by a 30-fold higher affinity for binding both DA and SST agonists, and associated with enhanced G protein and effector coupling to adenylyl cyclase. Indeed, hetero-oligomerization of D2R and SSTR5 led to synergy such that the binding affinity for the second radioligand was increased as a result of receptor occupancy by the first ligand. Given the endogenous co-expression of D2R and SSTR5 in striatal and cortical neurons, our results suggest that hetero-oligomerization of the two receptors may be one explanation for the reported enhancement of dopaminergic and somatostatinergic transmission induced by in vivo administration of SST or DA agonists. There are probably similar hetero-oligomers formed between other members of these two receptor families which could explain the full range of functional biological interactions between the two transmitter systems.

These results, obtained using pbFRET, indicate that hetero-oligomerization of SSTR5 and D2R is also induced by ligand binding, that ligand binding to either receptor can trigger hetero-oligomer formation, and that there are no preformed hetero-oligomers in the absence of ligand. The results of FRET analysis complement the pharmacological data in showing that hetero-oligomer formation induced by either SST or DA agonists correlates well with activated receptor function. There was, however, no strict correlation between the level of hetero-oligomer formation and the activity state of the receptor. Whereas pharmacological data demonstrated enhanced functional activity of the hetero-oligomer when occupied by both SST and DA ligands, no such synergy was found by FRET which showed the same level of hetero-oligomer formation for either agonist alone or both agonists applied together. Such dissociation is to be expected given that FRET will monitor only the presence of oligomers but not their activity state. Unlike SST and DA agonists which both promoted D2R/SSTR5 hetero-oligomer formation, the antagonists sulpiride and eticlopride produced modest or no hetero-oligomer formation as assessed by pharmacological and FRET analysis. Because there are no selective SSTR5 antagonists currently available, it was not possible to test the effect of antagonism at the SSTR5 receptor on D2R/SSTR5 hetero-oligomer formation. These results, however, suggest a model of GPCR hetero-oligomer formation in which agonist induces hetero-oligomer formation, with the hetero-oligomeric receptor complex simultaneously occupied by two ligands being the most active signaling form. Antagonists may act by preventing hetero-oligomer formation or by promoting the formation of inactive hetero-oligomers. Hetero-oligomer formation defines a new level of functional diversity in endogenous GPCR signaling.

### EXAMPLE 7

Hetero-oligomer Formation of Somatostatin Receptor (SSTR) Subtype1 and Subtype 5 is Ligand Concentration-dependent

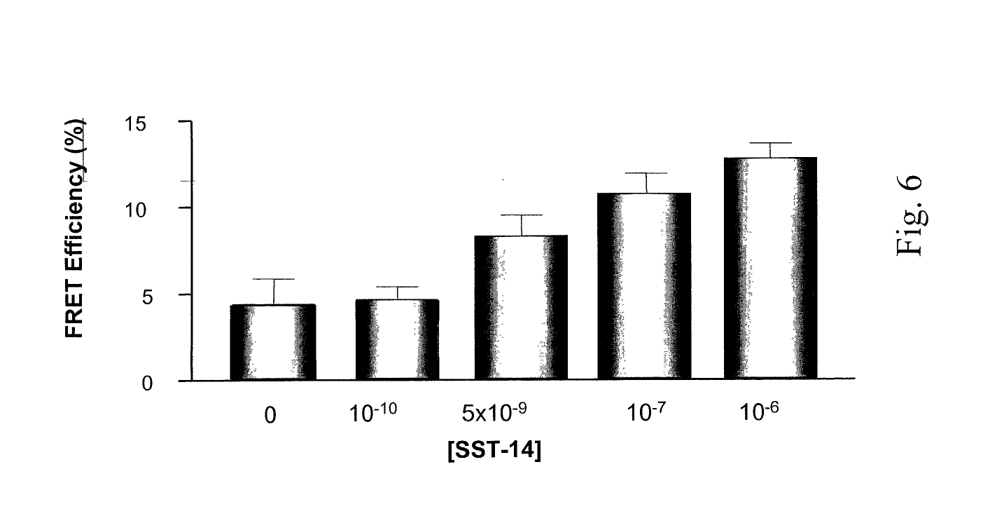

Dose-dependent increase in effective FRET efficiency by SST-14 in CHO-K1 cells expressing relatively low density of HA-SSTR5 and wild type SSTR1 (FIG. 6).

### EXAMPLE 8

Opioid Receptors (OR) and Somatostatin Receptors (SSTR) Interact as Hetero-oligomers to Form a Novel Receptor

Since ORs are structurally related to SSTRs and also exist as membrane hetero-oligomers, and because SST displays potent analgesic properties, SSTR5 and delta OR (DOR) were used as models to investigate hetero-oligomer formation as a molecular basis for the interaction between the SSTR and OR systems.

DOR and SSTR5 were colocalized with immunocytochemical techniques in distinct neuronal subsets in rat cerebral cortex using methods as described above.

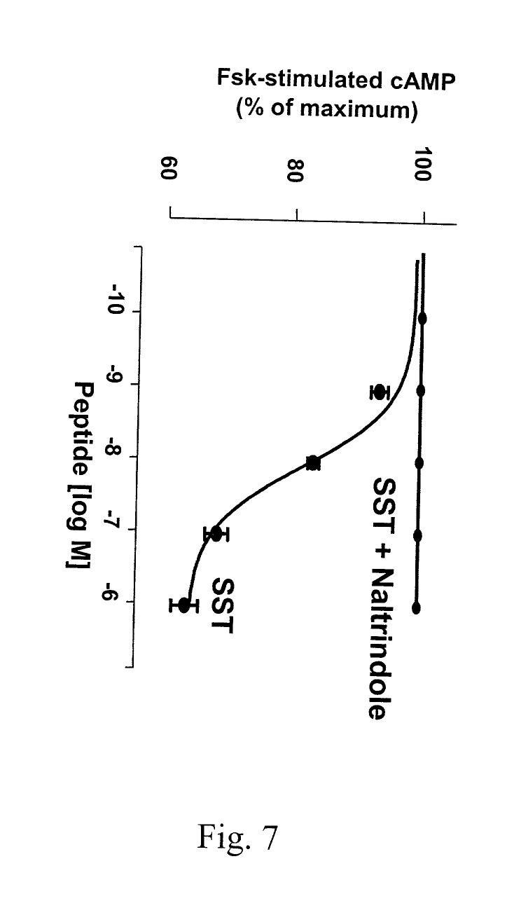

Hetero-oligomer formation was investigated by functional rescue of a binding competent, signaling-deficient C-tail deletion mutant of hSSTR5 (delta C-tail). Stable co-expression of delta C-tail with DOR in CHO-K1 cells followed by treatment with SST-14 induced dose-dependent, pertussis toxin sensitive inhibition of forskolin (FSK)-stimulated cAMP to a maximum of 70±3% at 10⁻⁶ M which was abolished by the DOR antagonist naltrindole (10⁻⁵ M) (FIG. 7).

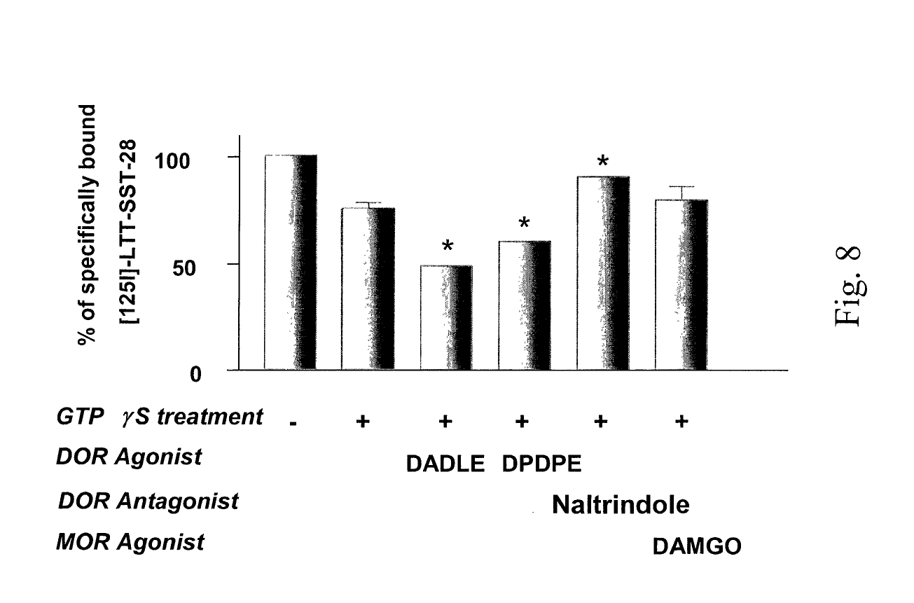

FIG. 8 illustrates the efficiency of G protein coupling in response to treatment with DOR agonists (DADLE ([D-Ala², D-Leu⁵]-Enkephalin)), DPDPE ([D-Pen^{2,5}]-Enkephalin), the DOR antagonist (naltrindole), or the Mu opioid receptor agonist (DAMGO ([D-Ala², N-MePhe⁴, Gly-ol⁵]-Enkephalin)). G protein coupling was assessed by the ability of GTPγS to inhibit binding of the SSTR5 radioligand [¹²⁵I] LTT SST-28 binding by the cotransfectants. In order to characterize G protein function, wild type hSSTR5 and DOR were stably coexpressed in CHO-K1 cells. Both the DOR agonists DADLE and DPDPE potentiated the inhibition of radioligand binding induced by GTPγs whereas the DOR antagonist Naltrindole reversed the effect of GTPγs and DAMGO was without effect. G-protein coupling efficiency was enhanced 2 fold by the DOR agonists DADLE, and DPDPE whereas the DOR antagonist naltrindole reduced coupling. These results show that Delta opioid agonists enhanced G protein coupling of the receptor complex induced by SST and that the DOR antagonist abolished it.

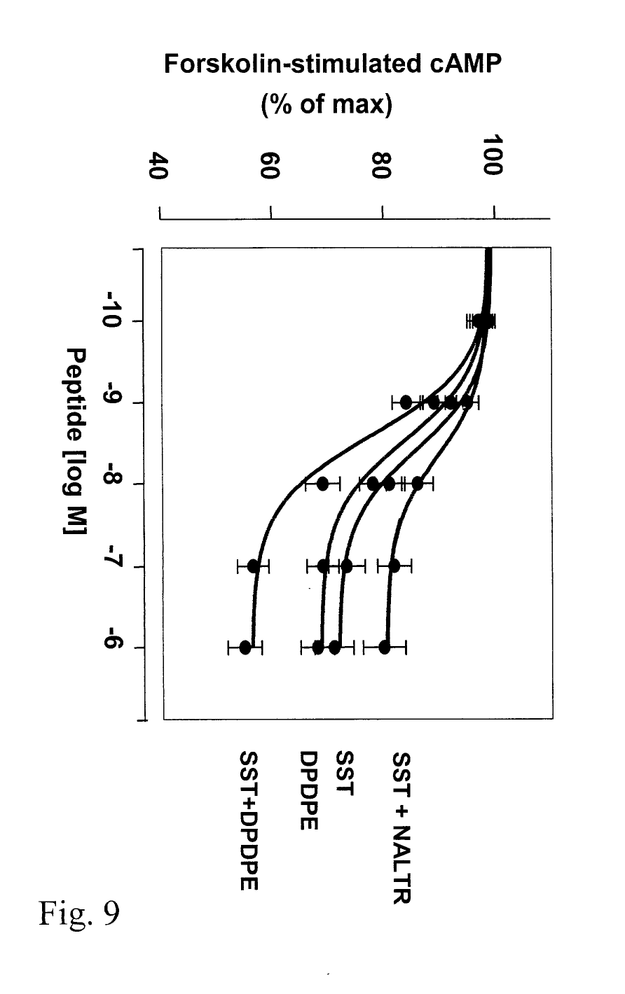

Treatment of the cotransfectants with SST-14 or DPDPE induced maximum 29±4% and 32±3% inhibition of FSK-stimulated cAMP respectively (FIG. 9). Simultaneous application of both agonists potentiated the cAMP inhibitory response to 45±3%. Naltrindole reduced SST-14 induced cAMP inhibition to 20±4%. These results show that DOR associates with hSSTR5 as a hetero-oligomer to form a novel receptor whose properties are distinct from those of the individual receptors, being characterized by enhanced functional activity when simultaneously occupied by both SSTR and DOR agonists (FIG. 9).

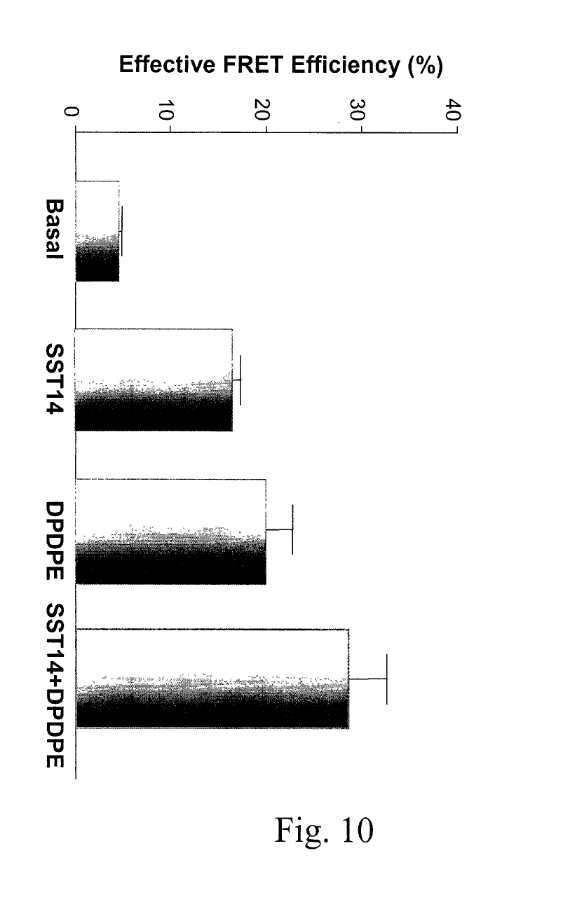

Direct evidence for the association of hSSTR5 and DOR as hetero-oligomers was obtained by photobleaching FRET microscopy or pbFRET as described in detail above in the Example concerning SSTR5 and D2R (FIG. 10). FRET depends on the property of an excited donor fluorophore to transfer its energy nonradiatively to an adjacent acceptor fluorophore. FRET occurs at distances of less than 100 angstroms and can be measured either directly as a change in signal intensity between donor and acceptor or by a competitive process called pbFRET. pbFRET depends on the principle that when a donor fluorophore is excited it will undergo a deactivation or photobleaching process due to radiative loss of energy to surrounding structures. If, however, there is an adjacent acceptor fluorophore, then FRET occurs and will competitively delay the photobleaching process. pbFRET is thus a lifetime measurement which measures time rather than direct spectroscopic signals and is ideal for studying FRET in intact cells cultured on glass slides. Cells cultured on microscope slides were treated with agonist, fixed and processed for immunofluorescence. pbFRET was performed by pixel analysis of plasma membrane to determine the photobleaching time constant of donor in the absence or presence of acceptor fluorophore.

Using CHO-K1 cells coexpressing HA-SSTR5 and delta OR, we observed a delay in photobleaching in the presence of acceptor fluorophore from 16.5 sec to 19.7 sec when SST-14 was added, confirming FRET and indicating close physical proximity of the two receptors in response to agonist treatment (FIG. 10).

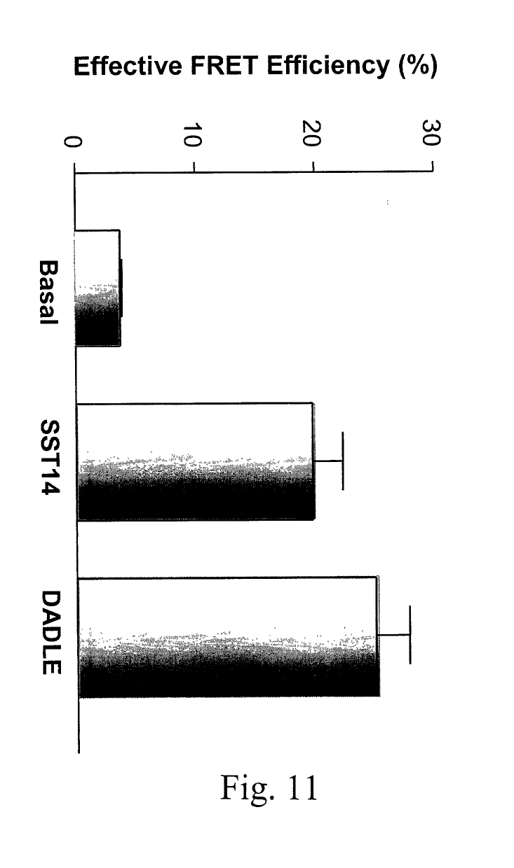

pbFRET microscopy was then used to study hetero-oligomer formation of endogenous DOR and SSTR5 receptors in primary cortical neurons using the same protocol developed for the CHO-K1 cell cotransfectants (FIG. 11). As in the case of CHO-K1 cell transfectants, there was low basal hetero-oligomer formation in the absence of agonist as indicated by a low effective FRET efficiency. Treatment with SST-14 or DADLE significantly increased FRET efficiencies to 19±4% and 24±3% as compared with the non-treated group (3±2%), indicating hetero-oligomer formation of SSTR5 and DOR in cortical neurons. This indicates that the two receptors are physically associated as hetero-oligomers in living cortical neurons (FIG. 11).

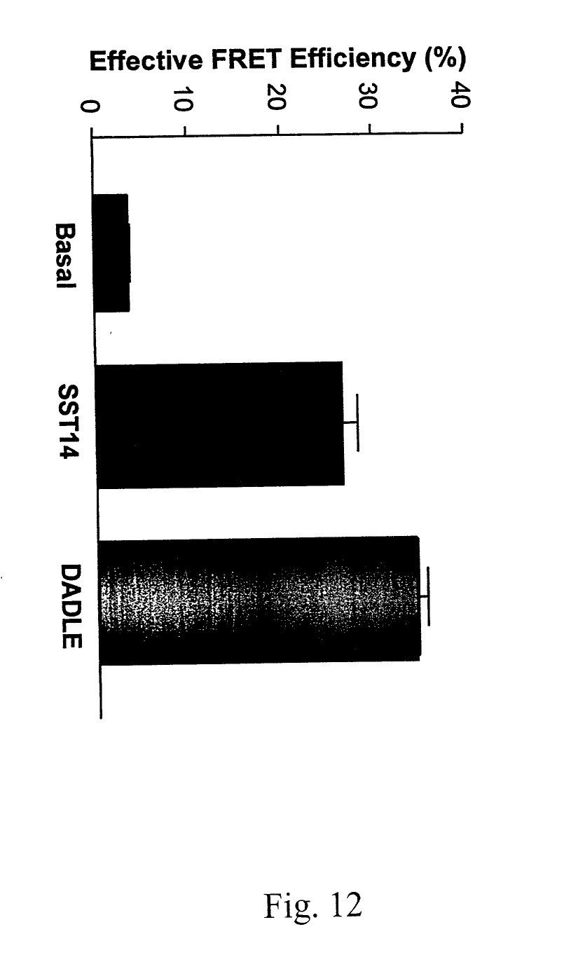

FIG. 12 illustrates a comparison of the FRET efficiencies obtained with SST-14 treatment compared to treatment with the DOR agonist DADLE in cultured primary striatal neurons. As in the case of CHO-K1 cell transfectants, there was low basal hetero-oligomer formation in the absence of agonist as indicated by a low effective FRET efficiency. Treatment with SST-14 or DADLE significantly increased FRET efficiencies indicating hetero-oligomer formation of SSTR5 and DOR in primary rat striatal neurons. pbFRET analysis of the two fluorescently labeled receptors in the plasma membrane of striatal neurons showed delayed photobleaching in the presence of acceptor fluorophore after SST-14 (10⁻⁶ M) treatment.

These results demonstrate hetero-oligomer formation for SSTR5 and DOR. These findings demonstrate that such hetero-oligomers exist in vivo as shown in the studies of cultured brain neurons, indicating that the results obtained using transfected cells are reflected in vivo.

### EXAMPLE 9

Multiple Somatostatin Receptor Subtypes (SSTRs) can Induce Apoptosis through Formation of Hetero-oligomers with SSTR3

Somatostatin (SST) induces variable apoptosis in tumor cells. When hSSTR1 through hSSTR5 are studied as monotransfectants in CHO-K1 cells, hSSTR3 is the only subtype that induces apoptosis. Since tumor cells endogenously express multiple SSTRs and often all five isoforms in the same cell, and since SSTRs are capable of forming functional hetero-oligomers with other family members, we investigated whether SSTRs other than SSTR3 could induce apoptosis through hetero-oligomer formation with SSTR3.

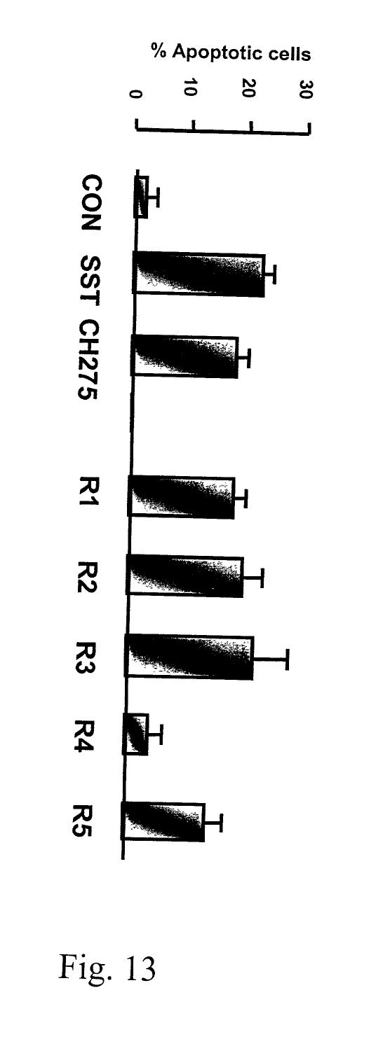

We selected for study MCF-7 human breast cancer cells which we showed by immunocytochemistry and reverse transcriptase-polymerase chain reaction (RT-PCR) to express SSTR1, SSTR2, SSTR3, and SSTR5 but not SSTR4. Cells were treated for 24-48 h with 10⁻⁹-10⁻⁷ M subtype-selective nonpeptide agonists (obtained from Merck). Apoptosis was assessed by TUNEL and Hoechst 33258 staining, as well as DNA fragmentation analysis by gel electrophoresis. Agonists for SSTR1, SSTR2, SSTR3, and SSTR5 each produced time- and dose-dependent apoptosis with the following rank order SSTR1 (25±3%), SSTR2 (23±4%), SSTR3 (18±5%), SSTR5 (18±4%) whereas the SSTR4 selective agonist was without effect (FIG. 13). Treatment of MCF-7 cells with antisense oligonucleotides to hSSTR3 (10 ug/ml) for 2 days prior to treatment with SST analogs abrogated apoptotic cell death induced by SSTR1, SSTR2, SSTR3 and SSTR5. This effect was not seen with sense SSTR3 oligonucleotide. In light of our recent finding that SSTRs can signal directly on the membrane through hetero-oligomer formation, the differential ability of SSTR1, SSTR2 and SSTR5 to induce apoptosis when co-expressed with SSTR3, but not when expressed as a monotransfectant, suggests that SSTR3 is an obligatory receptor for SST-induced apoptosis, and that other SSTR subtypes can also induce apoptosis through hetero-oligomerization with SSTR3. Indeed, we have confirmed by FRET analysis of CHO-K1 cells co-transfected with hSSTR3 and hSSTR2, as well as hSSTR3 and hSSTR5 that these two receptor combinations form hetero-oligomers.

All patents, publications and abstracts cited above are incorporated herein by reference in their entirety.

It should be understood that the foregoing relates only to preferred embodiments of the present invention and that numerous modifications or alterations may be made therein without departing from the spirit and the scope of the present invention as defined in the following claims.

We claim:

1. A cell transfected with at least two different G protein-coupled receptors.

2. The transfected cell of claim 1, wherein the at least two different G protein-coupled receptors form a hetero-oligomer.

3. The hetero-oligomer of claim 2, wherein each of the at least two different G protein-coupled receptors is from a different receptor family.

4. The hetero-oligomer of claim 2, wherein each of the at least two different G protein-coupled receptors is a different receptor subtype within a receptor family.

5. The transfected cell of claim 1, wherein the at least two different G protein-coupled receptors are at least two of somatostatin, opioid, adrenergic, adenosine, cannabinoid, dopamine, endothelin, muscarinic, serotonin, GnRH, chemokine, melanocortin, neuropeptide Y, GHRH, GHRP, TSH, LH, and FSH receptors, or combinations thereof.

6. The transfected cell of claim 1, wherein the at least two different G protein-coupled receptors are at least two of somatostatin, opioid, and dopamine receptors or combinations thereof.

7. The transfected cell of claim 1, wherein the cell is a neonatal cell, pituitary cell, neuronal cell, neuroendocrine cell, immune cell, endocrine cell, ovarian cell, kidney cell, tumor cell, or acinar cell.

8. The transfected cell of claim 1, wherein the cell is a AtT-20 cell, GH₃ cell, GH₄C₁ cell, PC12 cell, RinM5F cell, neuro2A cell, MCF7 cell, AR42J cell, Jurkatt cell, leukemic cell or myeloma cell.

9. A method of determining binding activity of a first ligand comprising:
measuring binding of a labeled second ligand to the cell of claim 1;
exposing the cell to the first ligand;
measuring binding of the labeled second ligand;
comparing the binding of the labeled second ligand before and after exposure to the first ligand.

10. A method of determining binding activity of a first ligand comprising:
measuring binding of a labeled second ligand to the cell of claim 1 when the first ligand is absent or present;
comparing the binding of the labeled second ligand in the absence and presence of the first ligand.

11. The method of claim 9, wherein membranes from the cell are used for binding the ligands.

12. The method of claim 10, wherein membranes from the cell are used for binding the ligands.

13. A method for determining interactions between a first GPCR and a second GPCR to form a hetero-oligomer, wherein the method comprises:
labeling the first GPCR with a donor fluorophore;
labeling the second GPCR with an acceptor fluorophore;
exposing the donor fluorophore to an excitation wavelength;
measuring efficiency of fluorescence resonance energy transfer between the donor fluorophore and the acceptor fluorophore.

14. A method of determining functional activity of a first ligand comprising:
comparing functional activity of a first ligand in a cell transfected with at least two G protein-coupled receptors to functional activity of the first ligand when a second ligand is present.

15. The method of claim 14, wherein functional activity is adenylyl cyclase activity, hydrolysis of inositol phosphate by phospholipase C, activation of phosphotyrosine phosphatase, conductance of voltage-gated calcium channels, activation of potassium channel currents, activation of MAP kinase, stimulation of mitogenesis, or induction of apoptosis.

16. A hetero-oligomer of G protein-coupled receptors expressed in a cell transfected with nucleic acids encoding for at least two different G protein-coupled receptor molecules, wherein the hetero-oligomer comprises at least two different G protein-coupled receptor molecules.
